(19) United States
(12) Patent Application Publication
(10) Pub. No.: US 2009/0294548 A1
(43) Pub. Date: Dec. 3, 2009

(54) Air Humidifier and Evaporation Mat Contained Therein

(76) Inventors: Stephan Geiger, Safenwil (CH); Karl Leu, Beinwil am See (CH); Robert Dubuis, Ipsach (CH)

Correspondence Address: HOLLAND & BONZAGNI, P.C., 171 DWIGHT ROAD, SUITE 302, LONGMEADOW, MA, 01106-1700, US

(21) Appl. No.: 12/223,756

(22) PCT Filed: Feb. 7, 2007
(86) PCT No.: PCT/CH2007/000064

§ 371 Date: Nov. 25, 2008

(30) Foreign Application Priority Data
Feb. 10, 2006 (CH) 216/06

(51) Int. Cl. A61L 9/04; B32B 27/02; B32B 3/28; B32B 5/02; B32B 27/04; B01F 3/04; F24F 6/04; B01D 1/00

(52) U.S. Cl. 239/6; 442/164; 442/171; 428/182; 428/152; 428/221; 442/141; 261/100; 261/106; 159/47.1

(57) ABSTRACT

Devices for the evaporation of liquid, in particular water, have an evaporation mat which is wetted with the liquid and on which the liquid evaporates. The evaporation mat comprises a textile fabric (1) having fibres, wherein the surface of the fibres is coated with a covering (2) which comprises a cured reaction product of a polyamine and a polyalkylene glycol etherified with end groups of the structure X—CH₂[CH(OR)]_{w}CH₂—, in which structure w is an integer from 0 to 1 and, when w is 0, X is a halogen, and, when w is 1, X is halogen and R is hydrogen, or X and R together are —O—. Preferably, the evaporation mat is a consolidated nonwoven which contains fibres made of a synthetic thermoplastic which are bonded to one another by means of a thermoplastic hotmelt glue at their intersection points. The devices are used for air humidification, for concentrating solutions, or for evaporative cooling.

The present invention concerns humidifiers and the evaporation mats contained therein.

Humidifiers are used in order to increase the humidity of air in closed rooms, in particular during the winter where the interior air is markedly too dry due to heating. In a humidifier the dry interior air to be humidified is driven over a wettened surface whereby water evaporates from the surface and thereby saturates the air partially with water vapour. In order to assist this evaporation process the humidifiers generally contain at least one so-called evaporation mat with a wettened structural surface as big as possible over which the air to be humidified is driven. The evaporation mats customarily consist of cellulose fibres, glass fibres, synthetic fibres or of metal wires, such as of aluminum. Frequently several evaporation mats which are appropriately shaped, in particular wave-shaped, are juxtaposed such that between two such juxtaposed layers of evaporation mats channels are formed which allow an improved passage of the air stream to be humidified.

In WO-A-99/32845 it is mentioned that such mats may be impregnated with a “wetting agent”, or that such mats may be provided with a “hygroscopic surface layer”. No examples, however, are given for the “wetting agent” or the “hygroscopic surface layer”.

On the other hand cured reaction products of a polyamine with a diglycidyl ether of a polyalkylene glycol have been used for several decades in the textile industry as antistatic coatings of textiles. Reference is made by way of example to U.S. Pat. No. 3,347,803.

The task to be solved by the present invention is to provide an improved device for humidifying air.

The task is solved according to the invention by a device for the evaporation of a liquid whereby the device has an evaporation mat which is wettened by the liquid and from which the liquid is evaporated, characterised in that the evaporation mat comprises a textile fabric with fibres whereby the surface of the fibres is coated with a coating comprising a cured reaction product of a polyamine and of a polyalkylene glycol etherified with end groups of the structure X—CH₂—[CH(OR)]_{w}CH₂—, in which structure w is an integer of 0 to 1 and, if w is 0, X means halogen, and, if w is 1, X means halogen and R means hydrogen, or X and R taken together mean —O—.

Preferred embodiments are according to the dependent claim.

It has been surprisingly found that these cured reaction products are suitable as hygroscopic coatings in evaporation mats assisting the evaporation of liquids, in particular of water. It has furthermore been found that such coatings adhere so strongly to the textile fabric that they are stable against gradual washing out. This is important for humidifiers since here the evaporation mat is continuously or intermittently wettened with water and there is the danger of washing out the coating if it does not adhere sufficiently strong. The resistance to washing out was also observed when the evaporation mats so coated were wettened with warmed-up water, i.e. with water of about 40° C. to about 80° C. It is hypothesized that this stability is caused by the crosslinking, which accompanies the curing of the reaction product.

The devices according to the invention contain firstly one or more evaporation mats as defined above. These are preferably clamped to a frame, such as from stainless steel or aluminum, in order to expose as much as possible of the geometric surface of the mat to the environment which is to take up the liquid to be evaporated. The environment which is to take up the liquid to be evaporated is in general gas-filled, in particular such environment is air-filled.

Furthermore the devices according to the invention may preferably comprise supply lines for the intermittent or continuous supply of liquid to be evaporated to the evaporation mat(s). A first example for such supply lines are nozzles, which are able to spray the clamped evaporation mat with the liquid. A further example for such a supply line is a tube, which is preferably mounted at the top edge of the evaporation mat and having a plurality of openings, which allow trickling the liquid to be evaporated from the top onto the evaporation mat from where it runs down the mat by the action of gravity.

In order to enhance the evaporation of the liquid the devices of the invention may preferably also comprise a means for circulating the gases, which causes the gaseous environment of the evaporation mat to circulate and thus provides for a continuous exchange of the gas-filled space near the surface of the evaporation mat(s). Examples for such means are fans and propellers.

The liquid to be evaporated may be any liquid which at the operating temperature of the device, which in general is about the ambient temperature around the device, has a notable vapour pressure but which does not yet boil at that temperature. Preferred examples for such liquids are all kinds of solutions of non-volatile substances in water or in organic solvents or in mixed aqueous/organic solvents, or pure water.

The devices according to the invention comprise a fibre-containing evaporation mat. As “fibres” are understood in the context of the present invention all kinds of elongated particles with a length much greater than the dimensions of the largest possible cross-sectional area measured perpendicularly to the longitudinal direction, which cross-sectional area is preferably constant. Examples of fibres are all true fibres of typically nearly constant circular and cross-section (these are preferred), or stripes cut out from foils, which therefore have an approximately rectangular cross-sectional area.

The fibres form the supporting framework of the textile fabric. The material from which they consist is not critical. It may be on the one hand a natural fibre (such as cotton, flax, hemp, jute, grey, ramie, silk, sisal or wool); they may also be inorganic fibres (such as fibres of glass, ceramic, alumina, carbon, metal, quartz or mineral wool); they may also be synthetic fibres (such as of polyester, viscose, PPS such as Ryton®, polyacrylnitrile, aramid such as Nomex®, Kevlar®or Kynol®, PVC or polyamide such as nylon). Fibres of a synthetic thermoplastic material are preferred. Examples for the synthetic thermoplastic material are:

i) polyesters, in particular polyethylene terephthalate or polybutylene terephthalate;
ii) polyolefins, in particular polyethylene, polypropylene and ethylene/α-olefin copolymers, whereby (C₃-C₈)-α-olefins are preferred as the α-olefin, and propylene is particularly preferred;
iii) polyamides, in particular nylon-6 or nylon-66; and
iv) thermoplastic polyurethanes.

Preferred according to the invention are polyethylene terephthalate and polypropylene.

The structure of the textile fabric is not critical. The textile fabric may be knitted, woven or a fleece. As a “fleece” is understood in the context of the present application a non-woven, non-knitted and non-plaited textile fabric. Preferably the textile fabric is an aerodynamically, hydrodynamically, electrostatically formed fleece or a fleece formed by extrusion (the latter is termed a spunbond); more preferably it is an aerodynamically manufactured fleece.

Preferably the textile fabric has a certain porosity. This porosity may be defined as the quotient A_{structural}/A_{geometric}, which is the quotient of total structural surface per unit of geometric surface of the textile fabric. This quotient A_{structural}/A_{geometric} may be calculated as follows:

$${A}_{structural } / {A}_{geometric} = 1000 \times G \times π \sum \frac{{x}_{i} \times {d}_{i}}{{T}_{i}}$$

in which formula G is the weight per unit of geometric surface of the textile fabric (in grams per square meter of its geometric surface), the sum runs over all i types of fibres present in the textile fabric, xᵢ is the mass fraction of the i-th fibre relative to the total mass of all fibres contained in the textile fabric, Ti is the linear density of the i-th fibre (in g per 1000 m of fibre length) and di is the diameter of the i-th fibre (in meters). The value of said quotient A_{structural}/A_{geometric} is preferably in the range of 10 to 50 (measured before any processing), more preferably it is about 20.

If the textile fabric is a fleece then the average length of the frame-forming fibres is preferably in the range of about 30 to 90 mm, more preferably about 40 to about 60 mm, and particularly preferably it is about 50 mm. The diameter of the fibres is preferably in the range of 10 to 100 μm, more preferably in the range of 15 to 30 μm, particularly preferably it is about 20 μm. The linear density range of the frame-forming fibres is preferably about 1 to about 17 dtex, more preferably about 3 to about 10 dtex and particularly preferably about 5 dtex (1 dtex means 10 km of fibre have a weight of 1 g).

The preferred textile fabric in the form of a fleece is more preferably a stiffened fleece. To achieve this the fleece may be adhered together at the fibre crossings of the frame-forming fibres by means of a suitable binder or, as preferred according to the invention, by means of thermoplastic hotmelt adhesive and by means of thermofusion. Suitable as the binder are thermoplastic binders, such as based on aqueous dispersions of polyacrylates and/or copolymers with polystyrene, ethylene-vinylacetate copolymers, polyvinyl acetate or vinyl chloride-ethylene-methyl methacrylate copolymer; duroplastic binders such as based on aqueous dispersions of acrylate polymers or of acrylate/olefin copolymers, which upon heating and under the influence of a hardener (such as a methylol group-containing compound, for example trimethylol propane or N-methylol carboxylic acid amides) cure with concomitant crosslinking. The thermoplastic hotmelt adhesive may be a copolymer. Preferably it is a copolyester. Examples for such copolyesters are on the one hand block copolyesters of soft segments derived of polyalkylene ether diols and/or long-chain (for instance C₅-C₁₂) aliphatic dicarboxylic acid esters with partially crystalline polybutylene terephthalate segments, and on the other hand copolyesters of aliphatic (C₂-C₁₀)diols (preferably of (C₂-C₄)diols, for example ethylene glycols, 1,3-propanediol, 1,2-propanediol, 1,4-butanediol, 1,3-butanediol, 1,2-butanediol or mixtures thereof) and of phthalic/isophthalic acids, whereby the molar ratio phthalic acid to isophthalic acid may preferably be in the range of 7:3 to 9:1.

The thermoplastic hotmelt adhesive which is preferably used for the preparation of a stiffened fleece has a melting point or melt range which is lower than the melting point or melt range of the material of the frame-forming fibres contained in the fleece. If the material of the frame-forming fibres is a thermoplastic synthetic material then its melting point or melt range may typically be in the range of about 130° C. to about 270° C. In contrast thereto the melting point or melt range of the holtmelt adhesive may typically be in the range of about 100° C. to about 150° C. That the “melt range lies in a range” is to be understood such that both the low boundary temperature value and the high boundary temperature value of the melt range shall be contained within the range in question. That a melt range is “lower” than another melt range has the meaning that the high temperature boundary value of the lower melt range is equal to or lower than the low temperature boundary value of the higher-lying melt range.

The fibres in the evaporation mat are coated with a coating which contains a cured and presumably crosslinked reaction product of a polyamine with an etherified polyalkylene glycol.

The preparation of the reaction product itself, i.e. before its curing, is described in the following.

As the polyamine all organic compounds are suited which contain at least two amino groups, which are primary or secondary. Preferably the polyamines are hydrocarbons substituted with two or more primary or secondary amino groups, whereby the hydrocarbon residue is straight-chain or branched, preferably straight-chain; preferably it has 2 to 8 carbon atoms in its main chain and the main chain may optionally be interrupted by one to three functional groups selected from —NH—, —O—, —NHCO—, —NHCONH— and —OCONH—, with the proviso that in the main chain two such functional groups being closest to each other are connected to each other over an optionally substituted alkylene spacer with a length of at least two carbon atoms. That the abovementioned hydrocarbon residue may be “branched” preferably has the meaning that a side chain selected from methyl and ethyl may be substituted onto the hydrocarbon chain. Similarly the term that the alkylene spacer may “optionally be substituted” means preferably that a substituent selected from methyl and ethyl is connected to the alkylene spacer.

Examples of classes of polyamines which can be used in the instant invention are:

a) linear 1,Ω-diaminoalkanes H₂N—(CH₂)ₘ—NH₂, whereby m is preferably 2 to 8, more preferably 2 to 4; examples are 1,2-diaminoethane, 1,3-diaminopropane, 1,4-diaminobutane, 1,5-diaminopentane or 1,6-diaminohexane;

b) the polyamines obtainable by reacting the 1,Ω-diaminoalkanes according to a), in particular those with the more preferred m of 2 to 4, with acrylonitrile and subsequent catalytic hydrogenation; these polyamines have, if the acrylonitrile is used in less than equimolar amount with respect to the amino groups, mainly the structure H₂N—(CH₂)₃—NH—(CH₂)ₙ—NH₂; if the acrylonitrile is used in equimolar amount or in excess the polyamines have mainly the structure H₂N—(CH₂)₃—NH— (CH₂)ₘ—NH— (CH₂)₃—NH₂;

c) the polyamines obtainable by reacting the 1,Ω-diaminoalkanes according to a), in particular those with the more preferred m of 2 to 4, with aziridine; these polyamines have, if the aziridine is used in less than equimolar amount with respect to the amino groups, mainly the structure H₂N—(CH₂)₂—NH—(CH₂)ₙ—NH₂; if the aziridine is used in equimolar amount or in excess the polyamines have mainly the structure H₂N—(CH₂)₂—NH— (CH₂)ₘ—NH— (CH₂)₂—NH₂;

d) the polyamines obtainable by reacting linear aliphatic 1,Ω-diisocyanates OCN—(CH₂)ₖ—NCO (whereby k is preferably 2 to 4) with the 1,Ω-diamines according to a); these polyamines have, if the diamine is used in less than equimolar amount with respect to the isocyanate groups, mainly the structure H₂N—(CH₂)ₘ—NHCONH—(CH₂)ₖ—NH₂; if the diamine is used in equimolar amount or in excess the polyamines have mainly the structure H₂N—(CH₂)ₘ—NHCONH—(CH₂)ₖ—NHCONH—(CH₂)ₘ—NH₂;

e) the polyamines obtainable by reacting linear aliphatic 1,Ω-bis-carboxylic acid halides XCO—(CH₂)ₚ—OCX (p is preferably 2 to 6) with the 1,Ω-diamines according to a) in an excess with respect to the acyl halide groups; these polyamines have mainly the structure H₂N—(CH₂)ₙ—NHCO—(CH₂)ₚ—CONH—(CH₂)ₘ—NH₂.

f) Further exemplary classes of polyamines may be obtained starting from glycols or polyetherdiols. The glycol (with the general structure HO—(CH₂)ₛ—OH, whereby s is preferably 2 or 3) or the polyetherdiol (with the general structure HO—(CHYCHZO)_{q}—H, whereby Y and Z are hydrogen or methyl, and q is preferably 2 to 4) may be firstly reacted with 1,Ω-dihalogenoalkanes in excess over the hydroxyl groups (i.e. more than one mol of dihalogenoalkane per mol of hydroxy groups) and then with excess ammonia. The 1,Ω-dihalogenoalkane has the structure X—(CH₂)ₜ—X, whereby X is selected from chlorine, bromine and iodine and t is preferably 2 to 4. Examples are 1,2-dibromoethane, 1,3-dibromopropane and 1,3-dichloropropane. If one starts with the glycol the polyamines so obtained have the structure H₂N—(CH₂)ₜ—O—(CH₂)ₛ—O—(CH₂)ₜ—NH₂; if one starts with the polyetherdiol they have the structure H₂N— (CH₂)ₜ—O—(CHYCHZO)_{q}—(CH₂)ₜ—NH₂.

A preferred polyamine is bis(2-aminoethyl)amine (=diethylene triamine).

In order to prepare the reaction product which is to be coated onto the textile fabric the above described polyamine is reacted with an etherified polyalkylene glycol. That the polyethylene glycol is “etherified” has the meaning that the two terminal hydroxyl groups of the polyalkylene glycol are etherified. The etherifying groups have, before undergoing the reaction with the polyamine, the structure X—CH₂—[CH(OR)]_{w}CH₂—, wherein w, X and R have the above mentioned meanings. The polyalkylene glycol itself may be either a pure substance of the formula HO—(CHYCHZO)ₙ—H, wherein v is an integer of preferably 1 to 4 and Y and Z independently from each other are methyl or hydrogen. In most cases the polyalkylene glycol is a mixture of homologous compounds of the formula HO—(CHYCHZO)ₙ—H which differ from each other in the number of repetitive units v and wherein Y and Z are also selected from hydrogen and methyl. This mixture of polyalkylene glycols is described not by v but by its mass-averaged molecular weight M_{w}:

$${M}_{w} = \frac{{\sum }_{i = 1}^{Z} {N}_{i} {M}_{i} {M}_{i}}{{\sum }_{i = 1}^{Z} {N}_{i} {M}_{i}}$$

in which formula i is an index running over all polyalkylene glycol homologues being present and Nᵢ and Mᵢ are the number of molecules and the molecular weight, respectively, of the i-th homologue. This averaged molecular weight M_{w} may be determined, as is customary in the art, on diluted solutions of the compound by light scattering measured according to the principle of “multi angle light scattering” (MALLS) with laser light. In the case of a mixture of homologues the M_{w} is preferably in the range of 200 to 1000 g/mol, more preferably in the range of 400 to 800 g/mol and particularly preferably it is about 600 g/mol.

In the formula HO—(CHYCHZO)ₙ—H preferably at least one of Y and Z is hydrogen, and more preferably both Y and Z are hydrogen. In the latter case the polyalkylene glycol is thus a polyethylene glycol.

The polyethylene glycol etherified with X—CH₂CH₂— (i.e. if w is 0) may be obtained, as is customary in the art, by direct preparation starting from X—CH₂CH₂—OH (whereby X has the above mentioned meaning) with an alkylene oxide of the formula

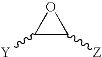

wherein Y and Z have the above mentioned meaning, in a desired multiple molar excess (relative to X—CH₂CH₂—OH), followed by a reaction with 1 equivalent X—CH₂CH₂—X (relative to X—CH₂CH₂—OH, whereby X has the above mentioned meaning) in the presence of an auxiliary base.

The polyalkylene glycol etherified with X—CH₂CH(OR)CH₂(i.e. if w is 1) may be obtained, if X means halogen and R means hydrogen, by reacting a polyalkylene glycol of the formula HO—(CHYCHZO)ₙ—OH, wherein v, Y and Z have the above mentioned meaning, or a mixture of such polyalkylene glycols, with an epihalohydrin of the structure

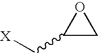

wherein X has the above mentioned meaning. This reaction may be done in analogy to example IX, lines 5-16 of U.S. Pat. No. 3,347,803.

The polyalkylene glycol etherified with X—CH₂CH(OR)CH₂(i.e. if w is 1) may be obtained, if X and R together should mean —O—, from the corresponding above described etherified polyalkylene glycol with X as halogen and R as hydrogen by reacting the latter for example with sodium aluminate (see lines 63-66 of example V of U.S. Pat. No. 3,347,803).

The reaction of the polyamine with the etherified polyalkylene glycol may be performed in analogy to known processes in an aqueous solution and in the presence of an auxiliary base such as NaOH. The ratio of etherified polyalkylene glycol to polyamine may be chosen preferably such that the molar ratio of reactive halide and/or epoxy groups to reactive hydrogen atoms bound to amino groups is in the range of about 4:7 to about 7:5. The concentration of the aqueous solution is preferably such that it comprises about 50 to about 75 percent by weight of reactants. The reaction temperature is preferably about 80° to about 150° C., whereby the heating is preferably carried out under reflux. The duration of the reaction may preferably be about 1 to about 4 hours.

The reaction product obtained from polyamine and etherified polyalkylene glycol may be used directly as a concentrated aqueous solution. In order to improve the stability on storage that solution may be adjusted to a pH of about 5.0 to about 6.0.

As such reaction products commercially available products falling under the definition according to the invention and, as mentioned in the introduction, have been used in the field of clothing for antistatic coatings of textiles, may also be used. Examples thereof being preferred according to the invention are the products Nonax 1166, Nonax 975 and Katax 570 commercialised by Henkel.

If desired the wetting of the textile fabric by the reaction product, when sprayed as a solution onto the textile fabric, may be improved by admixing tensides thereto. The amount of added tensides may typically be in the range of about 0.01 to about 0.5 percent by weight, relative to the dry solids content of the reaction product, whereby preferably an amount of tenside is added such that the solution of the reaction product, as ready to use for applying to the textile fabric, has a surface tension of at most 40 mN/m. The tenside is not critical. Preferred examples for suitable tensides are nonionic tensides (such as alkylphenol ethoxylates, fatty alcohol ethanolamines, fatty alcohol diethanolamines and alkyl fluoroethoxylates) or anionic tensides (such as alkyl sulfates, perfluoroalkyl carboxylates and perfluoroalkyl sulfonates).

For the preparation of the preferred stiffened fleeces the adhering of the fibres may be effected by spraying with the binder in question, which is typically in the form of an aqueous dispersion, and heating, which causes the crosslinking (and, if desired, simultaneously a wavy shape, see also below). If for the stiffening of the fleece a hotmelt adhesive is used, as is preferred for the invention, then this may be admixed already during the aerodynamic, mechanic or hydrodynamic preparation of the fleece in the form of a powder or in the form of fibres to the frame-forming fibres. Preferably, however, the hotmelt adhesive is employed in the form of a coating on the frame-forming fibres which fully or partially coats the frame-forming fibres. Such fibres coated with a hotmelt adhesive are known in the art as bicomponent fibres. The bicomponent fibres wherein the hotmelt adhesive fully coats the frame-forming fibres are also known as “coresheath” fibres. For the fibres wherein the hotmelt adhesive runs as a further strand in parallel to the frame-forming fibres and therefore only partially coats the latter the term “side-by-side” fibres has been used also in German-speaking countries. The preparation of fibres partially or fully coated with hotmelt adhesive is known and only reference is made to the corresponding literature in the art. Examples of commercially available “core-sheat” fibres which can be used in the instant invention are those with a polyester core (in particular polyethylene terephthalate) and a sheath of a copolyester as described above; trademarks are here Trevira® of Hoechst and Grilene® of Ems Chemie. Further examples are those with polypropylene or polyethylene terephthalate in the core and with polyethylene in the sheath; a corresponding trademark is ES® Fiber of Chisso Corporation. An example of a “side-by-side” fibre which is commercially available is ES® Fiber of Chisso Corporation, these have polypropylene as the frame-forming fibre and polyethylene as the hotmelt adhesive.

In a first preferred embodiment according to the invention of the textile fabric with a stiffened fleece “coresheath”fibres, optionally with addition or ordinary frame-forming fibres of a uniform synthetic material, are used. The weight ratio of ordinary frame-forming fibres to “coresheath” fibres may lie typically in the range of 0:100 up to about 80:20. The linear density of the “core-sheath” fibres is preferably equal to the linear density of the ordinary frame-forming fibres.

In another more preferred embodiment of the textile fabric with stiffened fleece ordinary frame-forming fibres of a unitary synthetic material admixed with fibres of a hotmelt adhesive are used. The weight ratio of ordinary frame-forming fibres to hotmelt adhesive fibres may typically be in the range of 40:60 up to about 80:20; preferably it is at least about 50:50. The linear density of the hotmelt adhesive fibres is preferably equal to the linear density of the ordinary frame-forming fibres.

The application of the above described reaction product onto the textile fabric is done preferably by spraying an aqueous solution of the reaction product or by dipping the textile fabric into that solution. Preferably the solution is sprayed. The aqueous solution's concentration of reaction product is preferably about 0.1 to about 10 percent by weight dry solids, relative to the weight of the solution, more preferably it is about 0.5 to about 5 percent by weight. The spraying or dipping solution is preferably adjusted to a pH in the range of 6 to 7 using an alkaline agent such as sodium carbonate. The wettening of the textile fabric with the spraying solution is preferably such that the reaction product is applied in an amount of 1 to 10 percent by weight dry solids, relative to the textile fabric.

If an enhanced flame proofness is desired then a customary but water-insoluble flame retardant in an amount of up to about 20 percent, relative to the solution, or up to 20 parts by weight per part of weight of dry solid of the reaction product, may be admixed to the aqueous spraying solution, whereby the amount of flame retardant to be added may be given from its efficacy and its solubility. Examples of flame retardants which can be used are aluminum trihydrate, red phosphor, polyphosphates, pentachlorophenol derivatives, antimony trioxide, melamine, melamine phosphate and water-insoluble, flame-retarding phosphonic acids and their esters. A preferred example of a flame retardant are phosphonic acid esters of the following formula:

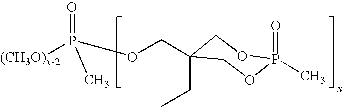

in which formula x is 0 or 1. Flame retardants of this type are commercialised for example by Albright & Wilson under the trademark Amgard®.

Before the curing of the reaction product the applied spraying solution is dried, preferably at normal pressure and at a temperature in the range of 80° C. to 150° C. during a time period of typically 1 to 3 minutes.

The curing of the reaction product on the textile fabric may typically be effected at a temperature of about 100° C. to about 180° C. during a time period of 5 seconds up to about 3 minutes, in analogy to the corresponding curing processes in the clothing industry, whereby the curing speed of the particular reaction product may also be taken into account. The temperature and duration of the curing is conveniently chosen such that the reactive groups of the etherifying residues of the polyalkylene glycol, i.e. the organically bound halogen atoms or the epoxy groups, react with the amine groups of the polyamine, such that a cured material is formed which is no longer soluble in the liquid to be evaporated.

If the textile fabric is a stiffened fleece then the above described curing of the reaction product is preferably carried out simultaneously and in one step together with the above described thermofusion of the frame-forming fibres, optionally with simultaneous mechanical deformation of the fleece with heated positive/negative forming tools. This allows, simultaneously to the adhering together of the fibres by thermofusion and the curing of the reaction product, to also impart the fleece a shape which has an advantageous effect on the flow of the gas stream which is to take up the water to be evaporated. The again cooled down, stiffened fleece retains the shape which has been imposed by the forming tools.

If the foregoing process is carried out on a fleece the fibres of which are essentially “core-sheath” fibres then the further advantage results that the reaction product not only cures with crosslinking but is also partially incorporated into the hotmelt adhesive forming the sheath of the fibres by thermosolisation, which further enhances the adhesion of the cured reaction product to the fleece.

A preferred shape for the evaporation mat, in particular also for the evaporation mats of the invention, is a quadratic or rectangular shape. Preferably it is wave-shaped such that it looks approximately like a quadratic or rectangular corrugated iron. Also preferred is here as the textile fabric a fleece which has been stiffened by means of a hotmelt adhesive. The direction of said waves runs preferably in a straight line and diagonally across the quadratic or rectangular evaporation mat, such as in an angle α of typically 20 to 60°, preferably about 30 to about 45° relative to a horizontal line, whereby in the case of a rectangular evaporation mat the longer side is to be preferably considered as the one lying transversally. The direction of the waves may be constant over the entire surface of the fleece; preferably, however, the direction of all waves changes at a given location to an angle of 180°-α with formation of a crease, such that each wave by itself looks like a V which is open either towards the top or towards the bottom. The direction of the waves might also change at regular intervals in a zigzag (for instance at intervals of 10 to 50 cm, depending on the field of use of the evaporation mat), whereby the changes of direction are alternating from said angle α to said angle 180°-α and vice versa.

Some of the evaporation mats to be used in the devices of the invention are novel themselves and thus also are objects of the invention. These are evaporation mats comprising a stiffened fleece with fibres of a thermoplastic synthetic material, which are in contact with each other at fibre crossing sites and which at these fibre crossing sites adhere together by means of a thermoplastic binder, a duroplastic binder or a thermoplastic hotmelt adhesive, whereby the surface of the fibres are coated with a coating comprising a cured reaction product of a polyamine with a polyalkylene glycol etherified with end groups of the structure X—CH₂[CH(OR)]_{w}CH₂—, in which structure w is an integer number of 0 to 1 and, if w is 0, X means halogen, and, if w is 1, either X means halogen and R means hydrogen, or X and R taken together mean —O—.

The devices for evaporating liquids according to the invention may be used for humidifying air, for concentrating up solutions or for refrigeration. For air humidification the liquid to be evaporated is primarily water, such as for instance normal tap water or desalinated water. For concentrating up solutions the liquid may be an arbitrary solution of a non-volatile substance in water, an organic solvent or a mixed aqueous/organic solvent. Exemplary non-volatile substances are inorganic or organic salts or organic substances such as sugar, wastes of sugar or dyes. Specific examples of solutions to be concentrated up are sea water, spent galvanisation baths or spent solutions of photography processing chemicals; landfill leachates, sewages from the chemical industry and spent electrolyte solutions from electrochemical processes such as the electrochemical raffination of noble metals, or solutions to be concentrated up of salts of the noble metals themselves. For the refrigeration water is commonly used as the liquid to be evaporated, such as in wet cooling devices. Refrigerants such as Freons or ammonia are also possible. The corresponding processes are also an object of the invention.

Important for the functioning of the invention is, as in the case of the prior art processes of evaporation, that the wettened evaporation mat is overblown with a stream of gas in which the partial vapour pressure of the liquid to be evaporated is lower than would be the partial vapour pressure of that liquid in that stream of gas if it was at a thermodynamic equilibrium with that liquid. Otherwise no evaporation of the liquid would take place.

The invention is now illustrated by specific embodiments with reference to the figures, in which figures:

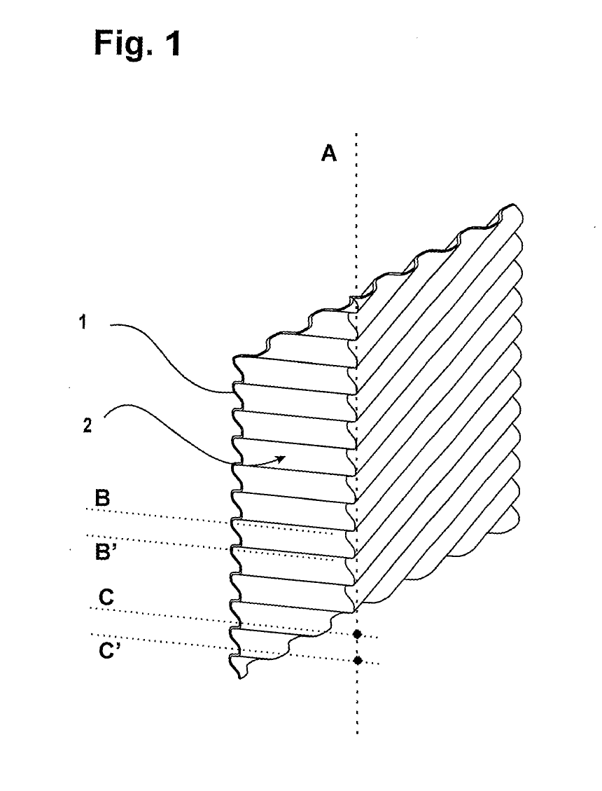

FIG. 1 shows a single evaporation mat according to the invention;

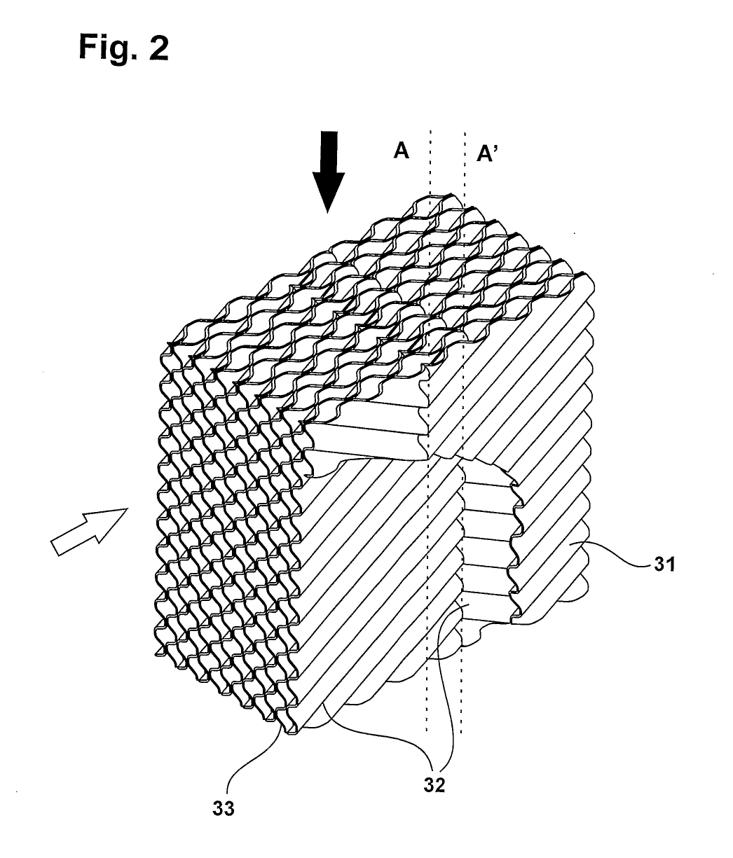

FIG. 2 shows a composite of several evaporation mats according to FIG. 1; and

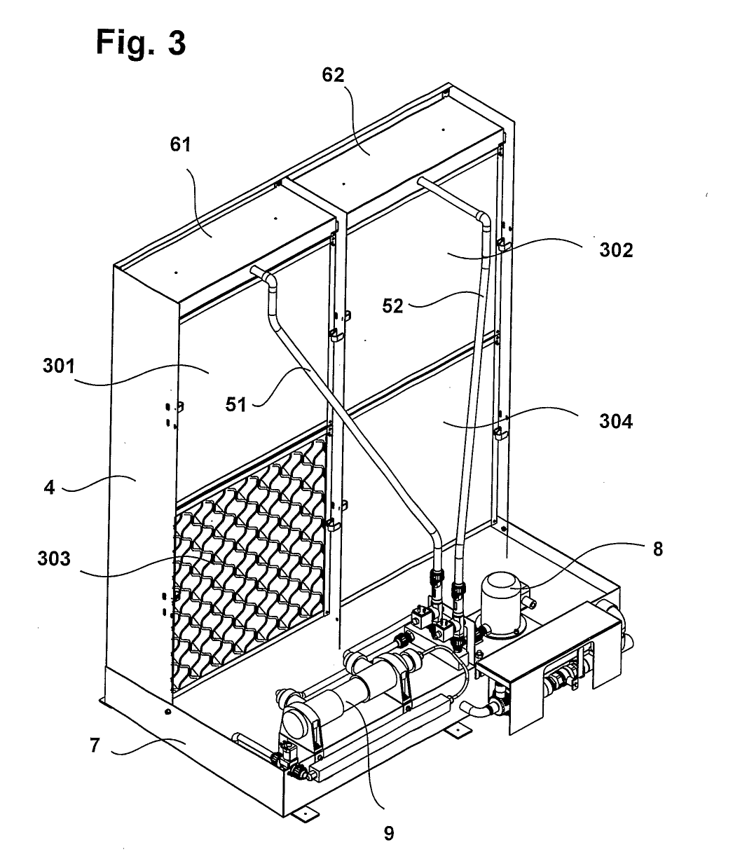

FIG. 3 shows an evaporation device according to the invention in the form of a humidifier, in which the supply of water is done by means of a tube, which trickles water from the top onto the evaporation mats.

FIG. 1 shows a rectangular evaporation mat according to the invention. The textile fabric 1 is an aerodynamically prepared fleece of frame-forming bicomponent fibres of the “core-sheath” type (Trevira 254 of Trevira GmbH, the core is polyethylene terephthalate, the sheath is copolyester, the fibre cross section is circular, the linear density is 2.2 dtex, the length of the fibres is 50 mm), the fleece having been pre-stiffened mechanically by needles. The cured reaction product of polyamine and etherified polyalkylene glycol is indicated as a surface coating 2 on the fleece. The evaporation mat has been deformed into a wavy shape and thus has approximately the shape of a corrugated iron. The valley lines of the throughs (only two of these have been designated as lines B, B′) and the summit lines of the crests (only two of these have been designated as lines C, C′) of the waves run in parallel to each other, whereby the valley lines (B, B′) of all throughs lie on an imaginary first plane (not shown in the figure) and the summit lines (C, C′) of all crests lie on a second imaginary plane (not shown in the figure) and whereby the first and second plane run in parallel to each other. The direction of the waves changes in a crease which has the shape of a V in angles of about 30° and 150° at a given location of the evaporation mat, whereby this location is defined by the intersection of the summit lines (C, C′) of the crests of that wave with an imaginary straight line A, which tangentially contacts all crests, and whereby the change of direction is identical for all waves. These intersections are shown as black circles. The preparation of the evaporation mat was carried out in this case as follows: A commercially available reaction product (Nonax 1166, Henkel) was used as an aqueous solution comprising, relative to the solution, 5 percent by weight (dry solids) of reaction product and 5 percent by weight of flame retardant (Flameguard HCA PW), and having been adjusted to a pH of 6 to 7 with 10% aqueous sodium carbonate solution. This solution was sprayed with an Airspray, Airmix or Airless system in an amount of 120 g per m² of fleece on the not yet stiffened fleece. The sprayed fleece was dried for 1.5 min at 130° C. (or 1 min at 150° C.). The dry fleece was placed in a deep drawing press with a heatable waved support and was pressed during 10 sec at 100° C. into the desired wavy shape and was stiffened using a heatable punch which matched the shape of the support, whereby simultaneously the reaction product of polyamine and etherified polyalkylene cured and simultaneously was partially bound to the hotmelt adhesive by thermosolisation.

FIG. 2 shows a composite of many evaporation mats of FIG. 1 (14 are shown). Only the first three ones are designated with reference signs 31, 32 and 33. For better understanding the top evaporation mat 31 has been shown in a partially cut-open fashion. The evaporation mats are juxtaposed in such a way that the V shapes of the waves point upwards or downwards in an alternating way, whereby the creases of the waves run on the intersections of their crests with imaginary lines A (mat 31) and A′ (mat 32), and whereby the lines A, A′ are in parallel to each other but are offset against each other. The stream of liquid is preferably supplied from the top into the composite (as indicated by the black arrow). The stream of gas which is to take up the liquid to be evaporated (i.e. typically the stream of air) is preferably driven laterally into the composite (as indicated by a white arrow), such that the liquid and the gas stream run past each other in an overcrossing manner.

FIG. 3 shows a device according to the invention for the evaporation of water, as would be typically used as a humidifier. This device is designed for being mounted into the ventilation shaft of a building which provides for the required circulation of the air to be humidified by means of ventilators. The device of the shown example has four composites 301, 302, 303 and 304 of evaporation mats according to the invention, these composites being similar to the ones of FIG. 2 and being mounted perpendicularly into a frame 4. In the composite 303 the wavy shape and the orientation of the individual evaporation mats is shown. The air stream to be humidified would preferably be driven from behind into the composites and would exit the composites at the front side of the composites. In each of the composites there are typically about 10 to 100 evaporation mats being juxtaposed one behind the other. Water is conducted over conducts 51, 52 into distributor casings 61, 62. In these distributor casings there is at the topside of the composites 301, 302 one single tube or several such tubes (not shown in the figure), which has (or have) a plurality of openings, which rinse the water onto the two composites 301, 302 and allow it to trickle down on them. The air current to be humidified and the stream of liquid are thus preferably driven in a crossflow through the composites. It was found that the coating on the evaporation mats is hydrophilic to such an extent that it is not necessary to wet all the evaporation mats of a composite; by wettening only a few evaporation mats eventually all evaporation mats contained in the composite are drawn full. The water applied onto the composites 301, 302 trickles down on these and then down on the lower composites 303, 304; excess water which is not evaporated is collected in the collecting tub 7. The tubes 51, 52 are provided with water by means of the water pump 8. The supply water may optionally be made free of bacterial by means of the UV sterilisation unit 9.

Instead of providing tubes, which allow trickling the water onto the composites 301, 302, 303, 304 from the top an arrangement of nozzles could also be used. These nozzles would conveniently be mounted on a vertical rack or grid, which is mounted vertically at an appropriate distance from the composites 301, 302, 303, 304.

1. A device for the evaporation of a liquid whereby the device has an evaporation mat which is wetted by the liquid and from which the liquid is evaporated, characterised in that the evaporation mat comprises a textile fabric with fibres whereby the surface of the fibres is coated with a coating comprising a cured reaction product of a polyamine and of a polyalkylene glycol etherified with end groups of the structure X—CH₂—[CH(OR)]_{w}CH₂—, in which structure w is an integer of 0 to 1 and, if w is 0, X means halogen, and, if w is 1, X means halogen and R means hydrogen, or X and OR taken together mean —O—.

2. The device according to claim 1, whereby the fibres are of a thermoplastic synthetic material.

3. The device according to claim 2, whereby the fibres comprise a thermoplastic synthetic material with a melting point or melt range in the range of 130° C. to 270° C., selected from the group of polyethylene terephthalate and polypropylene.

4. The device according to claim 1, whereby the textile fabric is a fleece.

5. The device according to claim 4, whereby the fleece has fibre crossings in which the fibres are in contact with each other and in which the fibres are adhered together by means of a thermoplastic binder, a duroplastic binder or a thermoplastic hotmelt adhesive.

6. The device according to claim 5, whereby the evaporation mat has a wavy shape in which the troughs and crests of the waves are in a straight line and run in parallel to each other, whereby the valley lines of all troughs are on an imaginary first plane and the summit lines of all crests are on an imaginary second plane and whereby the first and second imaginary planes are parallel to each other.

7. The device according to claim 6, whereby the direction of each wave changes at a given location of the evaporation mat with formation of a crease, whereby this location is defined by the intersection of the summit line of the crest of that wave with an imaginary straight line, which intersects the summit lines of all crests, and whereby the change of direction is identical for all waves.

8. The device according to claim 1, comprising one or more evaporation mats which lie atop of each other and which are adhered together.

9. The device according to claim 1, comprising one or more supply lines for the intermittent or continuous supplying of liquid to be evaporated to the evaporation mat(s).

10. The device according to claim 9, whereby the supply lines are nozzles, which spray the liquid onto the evaporation mat(s).

11. The device according to claim 9, whereby the supply line is a tube with a plurality of openings, which feed the liquid onto the evaporation mat(s).

12. The device according to claim 1, whereby the coating comprises a flame retardant.

13. The device according to claim 12, whereby the flame retardant is a phosphonic acid ester of the following formula: 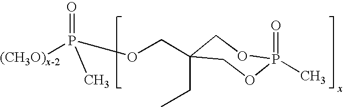
in which formula x means 0 or 1.

14. The device according to claim 1, whereby the textile fabric has a porosity in the range of 10 to 50, calculated as the quotient of total structural surface per unit of geometric surface.

15. An evaporation mat comprising a stiffened fleece with fibres of a thermoplastic synthetic material which are in contact with each other at fibre crossing sites and which at these fibre crossing sites adhere together by means of a thermoplastic binder, a duroplastic binder or a thermoplastic hotmelt adhesive, whereby the surface of the fibres is coated with a coating comprising a cured reaction product of a polyamine and of a polyalkylene glycol etherified with end groups of the structure X—CH₂[CH(OR)]_{w}CH₂—, in which structure w is an integer number of 0 to 1 and, if w is 0, X means halogen, and, if w is 1, X means halogen and R means hydrogen, or X and OR taken together mean —O—.

16. The evaporation mat according to claim 15, whereby the fibres comprise a synthetic material with a melting point or melt range in the range of 190° C. to 270° C., selected from the group of polyethylene terephthalate and polypropylene.

17. The evaporation mat according to claim 15, whereby the coating comprises a flame retardant.

18. The evaporation mat according to claim 17, whereby the flame retardant is a phosphonic acid ester of the following formula: 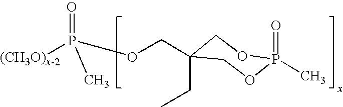
in which formula x means 0 or 1.

19. The evaporation mat according to claim 15, having a wavy shape, whereby the troughs and crests of the waves are in a straight line and run in parallel to each other, whereby the valley lines of all throughs troughs are on an imaginary first plane and the summit lines of all crests are on an imaginary second plane and whereby the first and second imaginary planes are parallel to each other.

20. The evaporation mat according to claim 19, whereby the direction of each wave changes at a given location of the evaporation mat with formation of a crease, whereby this location is defined by the intersection of the summit line of the crest of that wave with an imaginary straight line, which intersects the summit lines of all crests, and whereby the change of direction is identical for all waves.

21. A process for the evaporation of a liquid, whereby an evaporation mat as defined in claim 1 is wetted by the liquid and the wetted evaporation mat is overblown with a stream of gas in which the partial vapour pressure of the liquid to be evaporated is lower than would be the partial vapour pressure of that liquid in that stream of gas if it was at a thermodynamic equilibrium with that liquid.

22. The process according to claim 21, whereby the liquid is water and the evaporation of the water serves to humidify a gas-filled space, which is in the vicinity of the evaporation mat.

23. The process according to claim 22, whereby the gas-filled space contains air.

24. The process according to claim 21, whereby the liquid is a solution of a non-volatile substance in water, in an organic solvent or in a mixed aqueous and organic solvent and the evaporation of the liquid serves to concentrate the non-volatile substance in the liquid.

25. The process according to claim 24, whereby the aqueous solution is a salt solution, a sugar solution or a landfill leachate.

26. The process according to claim 21, whereby the liquid is water, an organic solvent or a mixed aqueous and organic solvent and the evaporation serves to refrigerate the liquid.

27. The process according to claim 26, whereby the aqueous solution is a coolant from a power plant's cooling tower or from a chemical process.

28. A process for the evaporation of a liquid, whereby an evaporation mat as defined in claim 15 is wetted by the liquid and the wetted evaporation mat is overblown with a stream of gas in which the partial vapour pressure of the liquid to be evaporated is lower than would be the partial vapour pressure of that liquid in that stream of gas if it was at a thermodynamic equilibrium with that liquid.
